(19) United States
(12) Patent Application Publication
(10) Pub. No.: US 2022/0175575 A1
(43) Pub. Date: Jun. 9, 2022

(54) METHODS AND DEVICES FOR PERSONAL CARE INCLUDING MENSTRUATION AND MICROBIAL INFECTIONS

(71) Applicant: Kimberly Jo Cull, Grove City, OH (US)

(72) Inventor: Kimberly Jo Cull, Grove City, OH (US)

(21) Appl. No.: 16/768,426

(22) PCT Filed: Nov. 30, 2018
(86) PCT No.: PCT/US2018/063326

§ 371 Date: May 29, 2020

Related U.S. Application Data
(60) Provisional application No. 62/593,206, filed on Nov. 30, 2017.

(51) Int. Cl. A61F 7/02; A61F 13/20

(52) U.S. Cl. CPC A61F 7/02; A61F 2007/0045; A61F 2007/0276; A61F 13/2045

(57) ABSTRACT

The invention disclosed herein generally relates to a methods and devices for personal care. The invention includes methods and devices for ameliorating microbial infections and for capture and disposal of menstrual fluid.

### CROSS REFERENCE TO RELATED APPLICATION

This application claims the benefit of U.S. Provisional Application No. 62/593,206, filed on Nov. 30, 2017. The entire contents of the foregoing are incorporated by reference.

### BACKGROUND TO THE INVENTION

Abnormal growth of normal microflora or pathogens such as filamentous yeast, fungi, viruses, bacteria, parasites, or protozoans can lead to health conditions on body surfaces and cavities.

### SUMMARY OF INVENTION

The invention disclosed herein generally relates to a methods and devices for personal care. Embodiments of the invention include methods and devices for ameliorating microbial infections and for capture and disposal of menstrual fluid. Microbial infections that are within the scope of embodiments of the invention as, for example, fungal nail infections, diaper rash, groin and scrotal rashes, infections that can contribute to pre-term birth, and the like.

Embodiments of the invention relate to a device for inhibiting abnormal growth of a pathogen on a body surface or cavity. The device can include a shell and a chamber, wherein the chamber is within the shell and can be capable of containing a freezable or cooling filler. The filler is capable of being frozen or rendered cooler and when the filler is frozen or rendered cooler and applied to body surfaces, the device can cool the body surface or cavity to a temperature that inhibits the growth of pathogens, changes pathogen morphology, and/or disrupts biofilms.

In some embodiments, the chamber has two compartments separated by a breakable barrier.

In some embodiments, the chamber can include a flexible material.

In some embodiments, the pathogen is a pathogen that causes a health condition selected from preterm labor, premature rupture of membranes, bacterial vaginosis, urinary tract infection, dermatophytosis, onychomycosis, tinea pedis, tinea cruris, rash associated with pendulous breasts, diaper rash, and/or the like.

In some embodiments, the body surface can be toe, foot, finger, hand, oral lips, vagina, labial lips, vulva, perineum, scrotum, penis, breast, panniculi, buttocks, and/or the like.

In some embodiments, the device can be configured to fit over a user's foot, wherein the device can include a strap for securing the shell to the user's foot.

Some embodiments of the invention relate to a method of inhibiting abnormal growth of a pathogen on a body surface or cavity. The method can include supplying a device comprising a shell and a freezable or cooling filler contained within a chamber within the shell; freezing the device until the filler is frozen or rendered cooler; applying the device to the body surface or cavity for a period of time sufficient to inhibit abnormal growth of the pathogen.

In some embodiments, the chamber can include a first and a second compartment separated by a breakable barrier.

In some embodiments, the first compartment can contain a first solution and the second compartment can contain a second solution; wherein the solutions combine when the breakable barrier is broken.

In some embodiments, the method can be used to treat, prevent, or cure a health condition such as bacterial vaginosis, urinary tract infection, sexually transmitted disease, dermatophytosis, onychomycosis, tinea pedis, tinea cruris, rash associated with pendulous breasts, diaper rash, and/or the like

In some embodiments, the method prevents pre-term birth.

Some embodiments of the invention relate to a device for capturing and disposing of menstrual fluid. The device can include a shell, a chamber, and an opening. The device can optionally include a sensor. The sensor can be capable of sensing menstrual fluid volume.

In some embodiments, the sensor can be capable of emitting a spontaneous signal to a monitor when the device is at full or near-full capacity.

Some embodiments of the invention relate to a method of capturing and disposing of menstrual fluid from a device. The method can include supplying a device including a shell, a chamber, and a sensor; inserting the device into a vagina during or before menstruation; wherein the sensor monitors volume of the menstrual fluid, wherein the sensor emits a signal to a monitor when the device is at full or near-full capacity.

In some embodiments, the device can be capable of being emptied without removing the device from the vagina.

In some embodiments, the device can be capable of being emptied by manual manipulation. Manual manipulation can be by using a ring, plug, tube, lever, valve, and/or the like which is attached to the device, wherein the device is unsealed and contents of the device flow out of the device by gravity when manually manipulated. For example, contents of the device flow out of the device by gravity when a ring is pulled; and the device is resealed when the ring is released. In some embodiments, alternatives to a ring can be employed, such as a tag or anything else capable of being grasped.

In some embodiments, the device is capable of being unsealed and resealed by an electronic signal.

### BRIEF DESCRIPTION OF THE DRAWINGS

FIG. 1 depicts a menstrual cup including a plug in the closed position.

FIG. 2 depicts a menstrual cup as the plug is being removed.

FIG. 3 depicts a menstrual cup including a plug that can be used as a removal tool.

FIG. 4 depicts a menstrual cup including a plug that can be used as a removal tool.

FIG. 5 depicts a menstrual cup including a plug with a loop wherein the plug is capable of removal by one finger via the ring. As depicted, the plug flexes so that it can be pushed into the menstrual cup and then returns to rest on a rim on the menstrual cup to prevent leakage.

FIG. 6 depicts a menstrual cup including a valve/lever wherein the valve/lever is in a closed position.

FIG. 7 depicts a menstrual cup including a valve/lever wherein the valve/lever is in an opened position.

FIG. 8 depicts a menstrual cup including a plug with a loop.

FIG. 9 depicts a menstrual cup including a tube wherein the tube is in a closed position.

FIG. 10 depicts a menstrual cup including a tube wherein the tube is in an opened position.

FIG. 11 depicts a menstrual cup including a magnetic hinged door in an opened position. The magnetic hinged door can be opened with a wand, as depicted. The wand can be a cylinder with rounded edges wherein one end can be a magnet to release or close the hinged door of the menstrual cup and the other end can be non-magnetic and shaped to push the hinged door to cup shut.

FIG. 12 depicts a menstrual cup including a magnetic hinged door in a closed position. The magnetic hinged door can be closed with a wand, as depicted. The wand can be a cylinder with rounded edges wherein one end can be a magnet to release or close the hinged door of the menstrual cup and the other end can be non-magnetic and shaped to push the hinged door to cup shut.

FIG. 13 depicts a menstrual cup including a hinged door in an opened position. The door can be opened electronically, for example, by a remote Bluetooth device.

FIG. 14 depicts a menstrual cup including a hinged door in a closed position. The door can be closed electronically, for example, by a remote Bluetooth device.

FIG. 15 depicts the onychomycosis slipper in the lateral view.

FIG. 16 depicts. the onychomycosis slipper in the ‘top’ view

FIG. 17 depicts the ‘the tinea pedis slipper in a ‘top’ view.

FIG. 18 depicts a glove for the hands.

FIG. 19 depicts a mitten for the hands.

FIG. 20 depicts the individual nail sleeve in a cross-sectional view.

FIG. 21 depicts data from experiments related to the effects of an intra-vaginal cooling device in mice.

FIG. 22 depicts data from experiments related to the effects of an intra-vaginal cooling device in mice.

FIG. 22 depicts data from experiments related to the effects of an intra-vaginal cooling device in mice.

FIG. 23 depicts data from experiments related to the effects of an intra-vaginal cooling device in mice.

### DETAILS OF INVENTION

The invention relates to methods and devices for personal care. In some embodiments, the device can be used in direct contact with a body surface or cavity. In some embodiments, the methods and devices relate to cooling treatments wherein the devices can inhibit the abnormal growth of pathogens and can disrupt biofilms on body surfaces. In some embodiments, the methods and devices relate to capture and disposal of menstrual fluid.

Some embodiments of the invention relate to a cooling device to treat and inhibit biofilms and the abnormal growth of pathogens on body surfaces and cavities comprising a shell, a freezable or cooling filler contained within a chamber within the shell, an optional strap, and optional flaps such that when the filler is frozen or rendered cooler and applied to body surfaces or cavities, the device cools the body surface or cavity to a temperature that inhibits the growth of pathogens, changes pathogen morphology and/or disrupts biofilms. Discussions herein of pathogen morphology are relevant inasumuch as some microbial pathogens exist in different morphological states. To use the yeast Candida albicans as an example, there is a non-pathogenic morphological state, known as the “bud” stage, in which C. albicans can exist in a body cavity such as the vagina with no deletrious effects. In fact, it is commonly found in the healthy vaginal microflora in the bud state. However, the genetically identical C. albicans can undergo a morpholigcal shift to an infectious (pathogenic) form known as they hyphal or filamentous stage. The filamentous hyphae grow in a manner that is infectious, uncomfortable, and even harmful to the patient, although the organism itself is genetically the same as the organism that previously lived benignly in or on the patient, in the bud stage. Accordingly, it is an object of the invention to provide methods and devices for causing a morphological shift in a pathogenic microbe, from the infectious morpology back to the non-infectious morphologyl.

Also disclosed is a device wherein the filler is at least one of a gas, liquid, solid, gel, or semi-solid. Likewise, the device can be configured wherein the chamber has at least two compartments, and optionally each compartment can be filled with a compound or substance that when the walls are broken, the substances mix to create a cooling substance from an endothermic reaction. The device can have a shape, size, and volume to conform to a body surface, such as a bridle, girdle, sling, bra and the like. The device can be designed to fit on the foot and cover the toes similar to a slipper, sandal, shoe, clog, or flat. Likewise the device can be designed to cover the hands and fingers similar to a glove or a mitten.

Additionally, the device can be configured so that it covers an individual finger and is similar to a digit cover or sleeve. Alternatively, the device can be configured so that it covers the oral lips or labial lips, vulva, perineum, and/or any other part of the body afflicted with or infected with a microbial infection susceptible to amelioration or cure by repeated cooling.

Embodiments of the invention also encompass a method of treating and inhibiting the abnormal growth of a pathogen, changing morphologies and disrupting biofilms on a body surface or cavity including: freezing or cooling the device, placing the device onto the affected body surface, thereby lowering the temperature on the body surface to a temperature that inhibits the growth of a pathogen and disrupts biofilms, removing the device when the cooling effect subsides, repeating freezing or cooling and applying it onto a body surface until the condition and until symptoms have abated or improved.

The cooling device simultaneously relieves the symptoms associated with these conditions such as itching, burning, redness, swelling, inflammation, pain, and nail discoloration and disfiguration. It may prevent foot ulcers common in diabetic patients which are precipitated by tinea pedis leading to bacterial cellulitis, osteomyelitis, amputations, and septicemia. The topical solutions and medications for onychomycosis (nail bed infections) do not penetrate into and through the hard nails very well, require months of therapy, and are often ineffective either due to noncompliance or suboptimal penetration.

The device acts via passive heat absorption resulting in tissue cooling, causing a cold-shock response (or inhibiting the heat shock response) by the pathogen, inhibiting further growth, allowing the nail to grow normally and the tissue to heal. Deeper penetration of the cooling effect is a result of keratinized tissue's thermal conductance properties. The device uses no systemic chemicals, is non-toxic, safe, and is faster-acting especially as it relates to tinea cruris and pedis. The itching and burning of these conditions are improved immediately and the ongoing infection is stopped faster without the skin irritation that topical anti-fungal creams routinely cause. Topical anti-fungal therapies destroy the fungal cell wall causing the release of enzymes which cause intense burning at the site, aggravating the symptoms they are designed to treat.

Devices can be for body surfaces such as the scrotum, penis, breasts, panniculi, buttocks, lips, or any other body surface afflicted with the pathogens described herein. Devices of embodiments of the invention are designed specifically for the appropriate size and shape of the body surface they are targeted to treat, to allow for the appropriate duration of therapy to effect a cure. A skilled artisan can recognize how to make and use adaptations of the concepts and embodiments disclosed herein to be useful for these various loci of infection.

These devices can include a filler or can be empty and then be filled with tap water or other suitable cooling/coolable substance by the user prior to use.

Some embodiments relate to methods for using the devices. In some embodiments, the devices are placed inclusively a freezer and allow the cooling material to cool substantially or to freeze completely solid or, in the case of mixing of chemicals, allow sufficient time to cause the filler to become very cold. The cooled or frozen devices can be applied to affected feet, hands, toes, fingers, or any affected body part. The devices can be left in place for a period of time, such as 20-45 minutes or until no further cooling sensation is experienced. This can be repeated until symptoms or conditions resolve or improve, or until a cure is achieved. Repeating cycles can be daily or more frequent such as, for example, every 18, 16, 14, 12, 10, 8, 6, 4, 3, 2, or 1 hour, or less, depending upon the duration of the contact period and clinical response.

Configurations for devices related to treating onychomycosis or tinea pedis are shown in the Figures. Devices to treat tinea cruris (jock itch) have a different shape due to the shape of the scrotum. Devices can be adapted according to the user, for example, in obese people, the intertriginous areas such as under large pendulous breasts and under panniculi are a different shape and flexibility which would guide material selection.

Likewise, a diaper or sling can be fashioned to conform to the user's buttocks, external genitalia, or external mouth or lips and filled with a cooling flexible gel or liquid for maximum contact with the afflicted body surface.

Embodiments of the invention also relate to a device configured to be inserted into a user's vagina for capture of menstrual fluid. The device can have a sensor to monitor the volume of menstrual fluid.

### Methods and Devices for Feminine Hygiene

### Introduction

The reasons for the increased use of re-useable menstrual cups are increased awareness, increased acceptance, less cost compared to alternatives, more fluid capacity, improved hygiene as compared with either tampons or pads, safer, and the unavailability of pads and tampons in developing countries. There is also the devastating societal impact from girls not attending school during menstruation in areas where pads are scarce.

Removing devices (menstrual cups) frequently from the vagina is manually difficult, painful, messy, time consuming, increases the risk of infection, and raises the chance of injury. With no ability to know if the device needs to be emptied, there is a chance that the device may overflow or not be filled with anything, in which case the user either has an accident due to overflow or the user subjects herself to unnecessary manual removal and reinsertion of the device.

Vaginal devices such as pessaries, intrauterine devices (IUD), medication impregnated vaginal rings (FEM RING and Nuva Ring), and fallopian tube plugs for sterilization are currently used safely and remain for prolonged periods of time in the vagina/uterus and during menstruation. Fallopian plugs are permanent, IUD last 1,3.5 years, FEM RING lasts 3 months, and pessaries can remain in the vagina for weeks or months.

There is no medical reason that a device in the vagina needs to be removed at regular intervals. Embodiments of the invention allow a device to remain ‘in-situ’ until it is no longer needed and then removed. For example, it would be medically and hygienically acceptable for a menstrual cup to be used with one insertion per cycle, and with one removal at the end of menstruation. This is far easier, less painful, and less risky than potentially removing the device 4 or 5 times each day for 5 to 7 days. Furthermore, removing this device in public places requires the user to go into a bathroom, remove the device over the toilet, clean up spills on the external part of the body, wash the device, and then replace the device in the vagina.

### EMBODIMENTS

Embodiments of the invention relate to a feminine hygiene device that can be inserted into the vagina during or prior to menstruation. The device can include a cup to hold vaginal and/or menstrual fluid. The cup includes an opening.

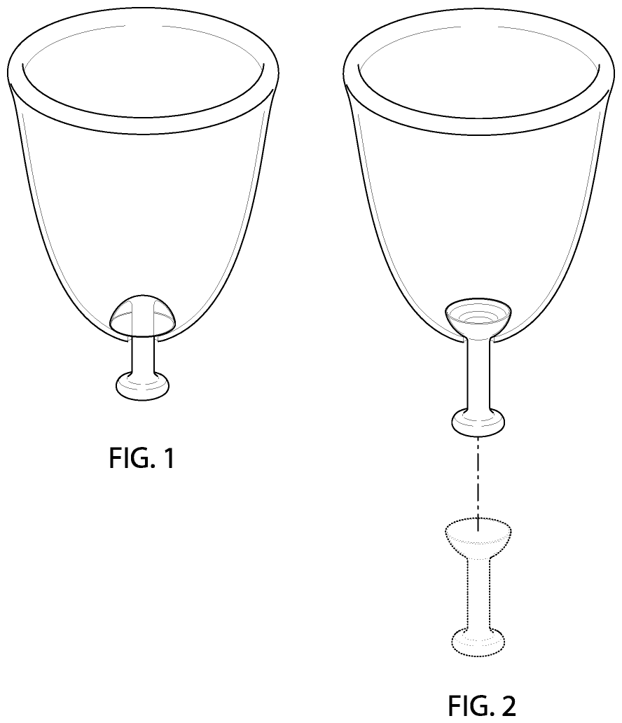

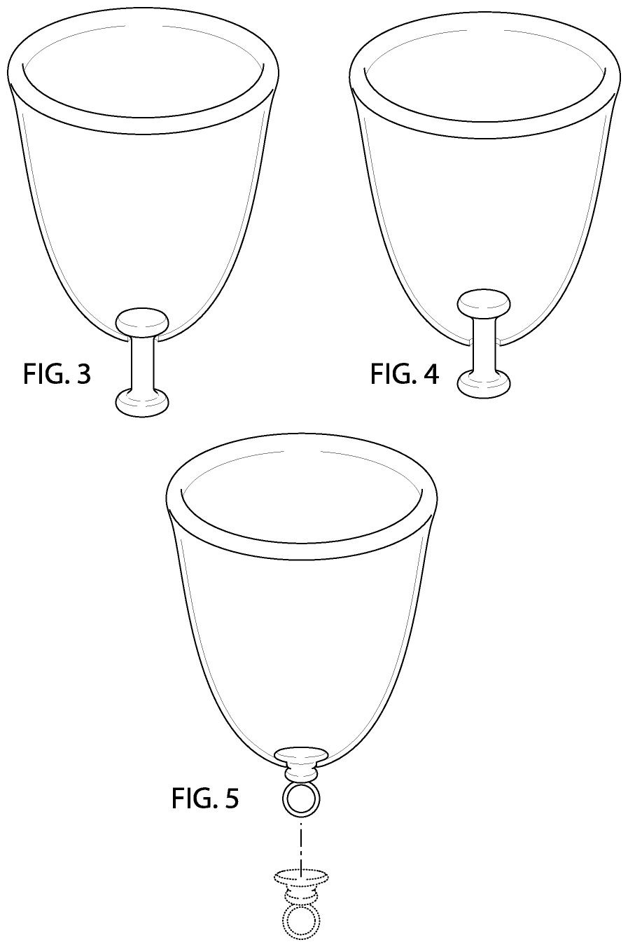

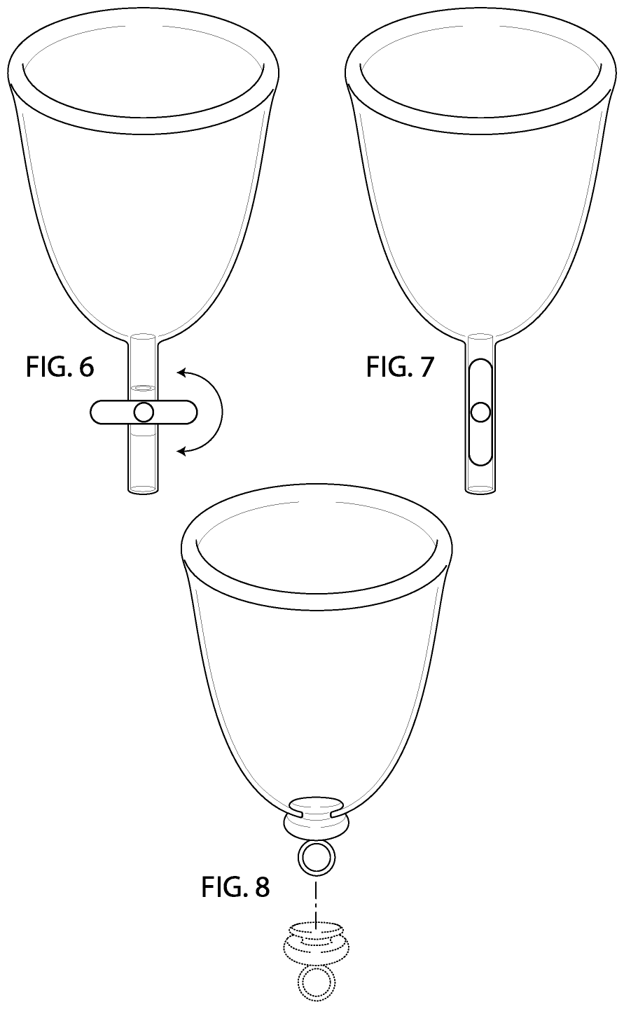

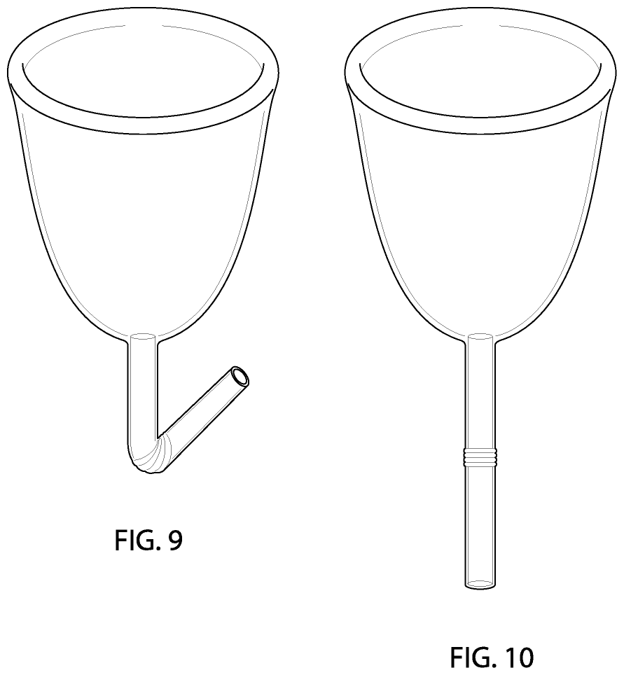

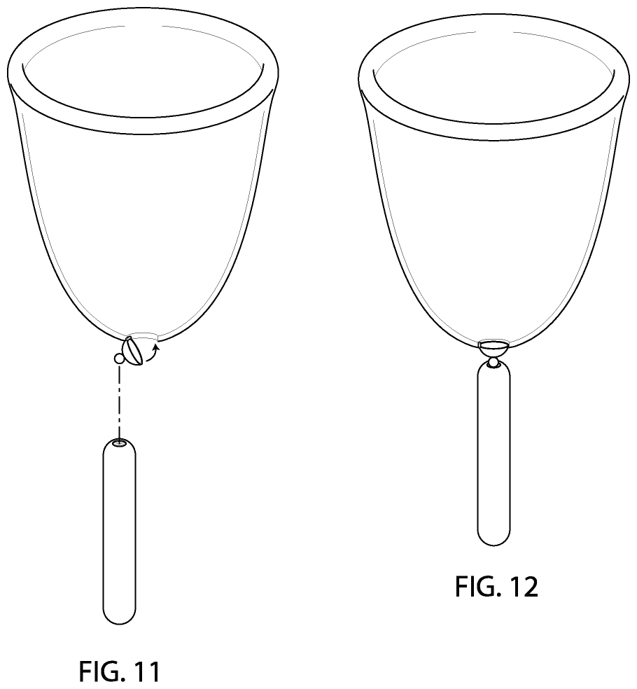

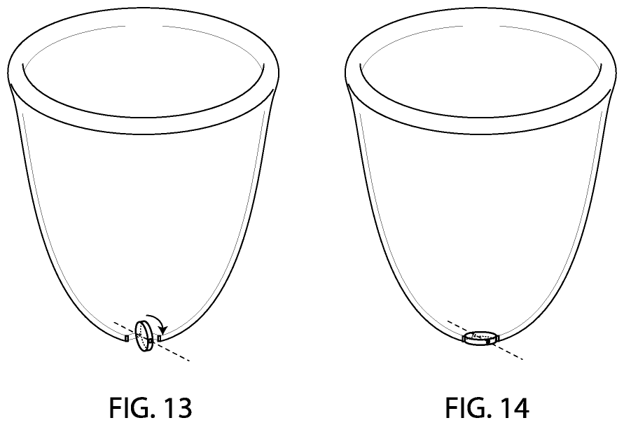

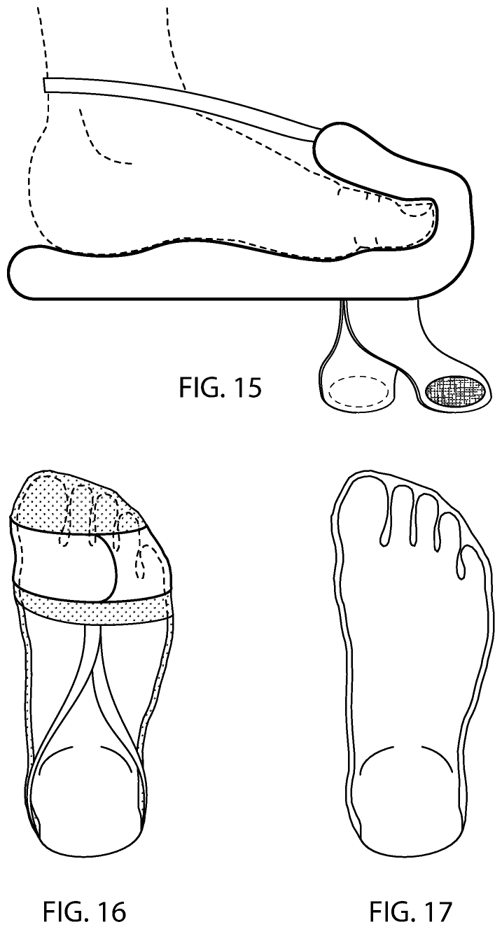

In some embodiments, the opening can be sealed with a removable plug. The plug can include a flexible dome-shaped component that can be inverted when inserted in the cup (see FIGS. 1 and 2). The inverted dome-shape can seal the cup and prevent leakage of vaginal fluids.

In some embodiments, the plug can be used as a removal tool.

In some embodiments, the plug can include a loop. The loop can allow for removal of the plug by a user's finger.

In some embodiments, the plug can be of a flexible material. The plug can be pushed into the cup and then can return to rest on a rim on the opening of the cup to prevent leakage.

In some embodiments, the device can include a valve and or lever to seal the opening. In some embodiments, when the valve/lever is in a horizontal position, the device is in a closed position so that the contents of the cup are contained and when the valve/lever in a vertical position, the device is in an opened position so that the contents of the cup can be released from the cup.

In some embodiments, the device can include a tube that can be bent. In some embodiments, when the tube is bent, the device is in a closed position and the contents of the cup are contained and when the tube is unbent, the device is in an opened position and the contents of the cup can be released from the cup.

In some embodiments, the device can include a magnetic hinged door to seal the opening of the device. The magnetic hinged door can be opened with a magnetic wand. The wand can be a cylinder with rounded edges wherein one end can be a magnet to release or close the hinged door of the menstrual cup and the other end can be non-magnetic and shaped to push the hinged door to cup shut.

In some embodiments, the device can include a hinged door can open and close. The door can be opened electronically, for example, by a remote Bluetooth device.

The device can be adapted to sense and notify a user that the device is at full or near-full capacity.

Some embodiments relate to emptying the contents of a device without removing the device from a body cavity.

In some embodiments, the device can include a component for inserting or removing the cup in or from a body cavity. The insertion/removal component can include a tool like a clamp that compresses the menstrual cup to a configuration and size that makes insertion easier and less painful and then releases the clamp once the menstrual cup is situated properly. This type of device can allow for easier removal as well. The insertion device can include a capsule and stalk with an internal plunger so that the cup can be compressed or folded and placed in the capsule, the capsule closed, placed in the vagina, and the plunger is deployed extruding the cup from the capsule for placement in the upper canal of the vagina. The purpose of the insertion device is to make the dimensions and shape of the cup more compatible with the user's anatomy.

In some embodiments of the invention, the menstrual cup has the capacity to sense when it is at full or near-full capacity and emits a spontaneous signal that notifies someone or something that the device is at full or near-full capacity, for example via transmission of a digital, electronic, or electromagnetic signal; or via episodic vibration of device, or the like. In some embodiments, the device can be emptied of its contents without removing the entire device from a body cavity. In some embodiments, the device can be under electronic, electromagnetic, or other suitable command that releases contents without manual manipulation. Likewise, the device can be manually manipulated to release its contents at any time. In some embodiments, the device can reseal itself manually or non-manually.

Methods and Devices for Preventing and/or Treating Preterm Birth

### Introduction

In 2004, more than 500,000 infants, or 12.5 percent of all infants, were born preterm, which is generally defined as birth at less than 37 completed weeks of gestation (CDC, 2005a). On the basis of new estimates provided in recent reports, the annual societal economic burden associated with preterm birth in the United States was in excess of $26.2 billion in 2005 (this estimate represents a lower boundary).

The percentage of preterm deliveries has risen steadily over the last 2 decades. Most of this increase has been among children born at 32 to 36 weeks gestation. Compared with infants born at term (37 to 41 weeks of gestation), preterm infants have a much greater risk of death and disability. Approximately 75 percent of perinatal deaths occur among preterm infants (Slattery and Morrison, 2002). Almost one-fifth of all infants born at less than 32 weeks gestation do not survive the first year of life, The infant mortality rate (IMR) per 1,000 live births for infants born at less than 32 weeks of gestation was 180.9, nearly 70 times the rate for infants born at between 37 and 41 weeks of gestation (Mathews et al., 2002)

The cause(s) of preterm birth is presently unknown, which makes finding an effective therapy for prevention difficult. According to the report Preterm Birth: Causes, Consequences, and Prevention by Richard E. Behrman, Adrienne Stith Butler, Editors, Committee on Understanding Premature Birth and Assuring Healthy Outcomes (2007), “the role of inflammation and its regulation during implantation and parturition should be studied. Specifically, perturbations to the immunologic and inflammatory pathways caused by bacterial, fungal, parasitic, and viral infections, along with the specific host responses to these pathogens, should be addressed.” Recently, vaginal fungal infections both symptomatic and asymptomatic have been evaluated as an etiology for preterm birth and premature rupture of membranes.

Recent studies have demonstrated a statistically significant reduction in preterm births among women who were treated with a topical anti-fungal azole, specifically clotrimazole for asymptomatic candidiasis before 20 weeks gestation. Using anti-fungal creams periodically throughout pregnancy has not been researched, primarily due to the unwillingness of clinicians and patients to subject the unborn fetus to repeated exposure to chemicals, especially if the woman is asymptomatic.

The purpose of this aspect of the invention is to introduce a non-chemical method to treat vaginal candidiasis and other microbial conditions of the vagina—both symptomatic and asymptomatic cases—with at least one application of an intravaginal cooling device, and with the possibility of repeated use periodically throughout pregnancy. Application of topical cooling to the vagina has been demonstrated in a limited clinical trial to cure vulvovaginal candidiasis (VVC). Biofilms, VVC, and bacterial vaginosis (BV) are all associated with the inflammatory process and associated immunological responses. Reducing inflammation, microbial antigen exposure, and microbial infections are the subject matter for this invention.

Various conditions including BV are known to be associated with premature delivery and premature rupture of membranes. Both BV and VVC are associated with biofilms which make treatment difficult as a result of suboptimal penetration of an anti-microbial or anti-fungal agent in the biofilm. This results in a poor cure rate and high recurrence rate for both BV and VVC.

Furthermore, during pregnancy, the frequency of urinary tract infections (UTI) is increased. As a result, antibiotics are administered during pregnancy to treat the UTIs. Antibiotics shift the microflora of the vagina which changes the pH to a less acidic environment. This encourages the development of VVC. Likewise, elevated progesterone levels in pregnancy and the thermogenesis created by the metabolism of the growing fetus increase core body temperatures, and this also predisposes a pregnant women to VVC, including both symptomatic and asymptomatic cases.

Topical anti-microbial and anti-fungal creams contain ingredients that contribute to biofilm formation. Embodiments of the present invention are aimed at reducing the occurrence of preterm birth and premature rupture of membranes via prevention and treating ongoing premature labor or rupture of membranes without the need for chemicals or in conjunction with less toxic types or dosages of chemicals if necessary. This is achieved by applying a topical cooling device to the intra-vaginal space, with or without cervical contact, at least once, and possibly multiple times.

### Embodiments

Disclosed herein are method and devices for preventing or treating preterm birth, premature labor or contractions, premature cervical dilatation or cervical incompetence, or premature rupture of membranes by the administration of a topical cooling device to a body cavity or surface for a sufficient frequency and duration of time to change microbial pathogen morphology, induce microbial dormancy, reduce biofilms, stop contractions, stop cervical dilatation, or to prevent the occurrence of preterm birth or premature rupture of membranes before 37 weeks gestational age.

The methods and devices disclosed herein can be useful in treatment or prevention of preterm birth, treatment or prevention of preterm labor, treatment or prevention asymptomatic cervical dilatation (cervical incompetence), and treatment or prevention of premature rupture of membranes.

In some embodiments, an intra-vaginal cooling device also touches the cervix.

Likewise, in some embodiments, topical cooling is applied to the external surface of the abdomen overlying the uterus at the same time. Accordingly, in some embodiments the cooling accomplished by the devices and methods disclosed herein can act not only as an anti-fungal but also to reduce inflammation, further stabilizing the prenatal environment and promoting the benefits of embodiments of the invention.

The cooling of the vagina, cervix, and/or uterus according to this disclosure can therefore prevent or treat:

(1) premature births (<37 weeks)
(2) premature labor with or without cervical dilatation or effacement (<37 weeks)
(3) premature dilatation or effacement (thinning) without labor (cervical incompetence)
(4) premature rupture of membranes with or without contractions, with or without cervical dilatation or effacement (<37 weeks)

Some devices and methods useful for this reduction in premature births are disclosed in U.S. patent application Ser. No. 13/637,496, titled TREATING MEDICAL CONDITIONS IN BODY CAVITIES, filed on Sep. 26, 2012; and Ser. No. 14/059,843, titled TREATING CONDITIONS CAUSED BY ABNORMAL GROWTH IN BODY CAVITIES, filed on Oct. 22, 2013. Each of these patent applications is incorporated herein by reference in its entirety.

Methods and Devices for Preventing and/or Treating Diaper Rash

### Introduction

Rashes that are directly or indirectly caused by the wearing of diapers: This category includes dermatoses, such as irritant contact dermatitis, candidal diaper dermatitis, malaria, intertrigo, and granuloma gluteale infantum

The precise cause of most diaper rashes is not clearly defined. A combination of factors that contribute to diaper rash includes wetness, friction, urine and feces, and the presence of microorganisms. This skin region features numerous folds and creases, which present a problem with regard to both efficient cleansing and control of the microenvironment.

The main irritants in this area are fecal proteases and lipases, whose activity is increased greatly by elevated pH. An acidic skin surface is also essential for the maintenance of the normal microflora, which provides innate antimicrobial protection against invasion by pathogenic bacteria and yeasts. Fecal lipase and protease activity is increased by diarrhea in the previous 48 hours

The wearing of diapers causes an increase in skin wetness and pH. Prolonged wetness leads to maceration (softening) of the stratum corneum, the outer, protective layer of the skin, Weakening of its physical integrity makes the stratum corneum more susceptible to damage due to friction from the surface of the diaper and local irritants.

The normal pH of the skin is between 4.5 and 5.5. When urea from the urine and stool mix, urease breaks down the urine, decreasing the hydrogen ion concentration (increasing pH). Elevated pH levels increase the hydration of the skin and make the skin more permeable.

High soap pH values encourage propionibacterial growth on skin, whereas synthetic detergents with a pH of 5.5 did not cause deleterious changes in the microflora.

Once the skin is compromised, secondary infection by Candida albicans is common. Between 40% and 75% of diaper rashes that last for more than 3 days are colonized with C albicans. Candida has a fecal origin. Antibiotics such as amoxicillin were found to increase the colonization by Candida and worsened the diaper dermatitis.

Polymicrobial growth is documented in at least half of diaper rash cultures.

Staphylococcus species are the most commonly grown organisms, followed by Streptococcus species and organisms from the family Enterobacteriaceae. Nearly 50% of isolates also contain anaerobes. This can result in a bacterial cause for diaper rash. Granuloma gluteale infantum is rare but is thought to be the result of an unusual inflammatory response to prolonged irritation, candidiasis or fluorinated corticosteroids.

Morbidity for the child mostly is in the form of pain and itching in the affected areas. The incidence is reported as anywhere from 4-25% and in one study, diaper rash accounted for nearly 20% of pediatric office visits.

Candida diaper rash should be suspected in all rashes lasting more than 3 d (Candida is isolated in 45-75% of such cases). It is usually painful as parents report severe crying during diaper changes or with urination and defecation as the excoriated and inflamed skin reacts to the process.

If candidal infection is suspected, topical ointments or creams, such as nystatin, clotrimazole, miconazole, or ketoconazole can be applied to the rash with every diaper change. Other products used are combination products such as miconazole and hydrocortisone, combination product of nystatin/benzalkonium chloride/dimethicone/hydrocortisone preparation. In some severe cases, oral nystatin has been prescribed. Ciclopirox has been used and found effective. Burning, redness, or itching at the application site may occur according to the first statement listed in the side effects section of the prescribing information. Furthermore blistering/swelling/oozing at the application site was rare, but not impossible as they state that the majority of people did not experience severe reactions.

A recent study examined the efficacy and safety of sertaconazole (another azole) cream (2%) in diaper dermatitis candidiasis and concluded that sertaconazole cream may be considered a new alternative for diaper dermatitis candidiasis treatment. However, adverse events associated with topical application of sertaconazole cream were mostly cutaneous-related and included contact dermatitis, dry or burning skin, application-site reaction, eczema, itch and skin tenderness. These chemicals have ingredients that can also cause skin sensitivity and result in the release of microbial contents due to drug's mechanism of action against the microbe. Likewise, the table below lists the other types of ointments and products that have been used to protect the skin and possibly reduce the risk of diaper rash without clear proof that they are effective and with the possibility that their properties actually encourage yeast or microbial growth i.e., humectant or brings water to the surface. Keeping the area dry, not moist is the goal to prevent microbial overgrowth. Some of these listed chemicals are counterintuitive or contraindicated. Methyparabens, cetyl alcohol, and isopropyl alcohol are just a few of the proven skin irritants and should be avoided in this fragile population.

Several products are available for the care, management, and maintenance of skin integrity. The following are examples of ingredients frequently found in skin care products.

| | |
|---|---|
| Petrolatum | Skin protectant, water repellant, a barrier |
| Zinc oxide | Skin protectant, soothes irritated skin |
| Dimethicone | Skin protectant |
| Vitamins A and D | Skin conditioner |
| Karaya | Viscosity modifier and absorbs moisture |
| Mineral oil, lanolin, glycerin | Emollient, softens and soothes irritated skin, a lubricant; |
| | Humectant, hygroscopic (brings water to the surface of the skin |
| | producing a moisturizing effect) |
| Vitamin E acetate | Skin conditioner |
| Isopropyl palmitate | Skin conditioner |
| Purified water | Diluent |
| Chloroxylenol (PCMX) | Antimicrobial, kills or inhibits bacteria |
| Isopropyl alcohol | Antimicrobial |
| Miconazole nitrate | Antifungal |
| Carboxymethylcellulose | Viscosity modifier |
| sodium | |
| Methyl glucose dioleate | Emulsifier, added to water-oil preparations to prevent the oil |
| | from separating from the water |
| Stearate acid | Emulsifier |
| Butylparaben | Preservative, prevents breakdown of product and destroys or |
| | prevents growth of bacteria |
| Methylparaben | Preservative |
| Triethanolamine | pH adjuster (normal pH of skin is 4.5-5.5) |
| Aminomethyl propanol | pH adjuster |
| Cetyl alcohol | Emollient and thickening agent |

Adapted from Pediatr Nurs. 2004 November-December; 30(6): 467-70.^{[14]}

Since chemical treatments take days to work, can cause the symptoms to worsen initially, possibly increase the risk of microbial resistance, with the associated risk of increased virulence, it is the present inventions intention to treat the diaper rash faster, more safely, and more effectively with a cooling device that can be placed or applied intermittently for 30 minutes to one hour and repeated as necessary to cure the condition. The soothing cooling process stops symptoms immediately as topical cooling has been known for decades to be anti-pruritic, analgesic, and anti-inflammatory.

### EMBODIMENTS

The methods and devices disclosed herein can be useful in treating and preventing the occurrence of diaper rash associated with microbial overgrowth by the application of a cooling diaper-shaped device that is soft and flexible for placement around the buttocks, groin, upper thighs, perineum, or any combination thereof.

Any configuration of a soft, flexible cooling device that can be placed on or wrapped around the affected area either via direct skin contact or applied over an existing diaper.

### Treating or Preventing Tinea Cruris

### Introduction

The most common etiologic agents for tinea cruris include Trichophyton rubrum and Epidermophyton floccosum; less commonly Trichophyton mentagrophytes and Trichophyton verrucosum are involved. Tinea cruris is a contagious infection transmitted by fomites, such as contaminated towels or hotel bedroom sheets, or by autoinoculation from a reservoir on the hands or feet (tinea manuum, tinea pedis, tinea unguium). The etiologic agents in tinea cruris produce keratinases, which allow invasion of the cornified cell layer of the epidermis. The host immune response may prevent deeper invasion. Risk factors for initial tinea cruris infection or reinfection include wearing tight-fitting or wet clothing or undergarments. Dermatophytosis accounts for approximately 10-20% of all visits to dermatologists.

Tinea cruris has a worldwide distribution but is found more commonly in hot humid climates. Tinea cruris is 3 times more common in men than in women. Adults are affected by tinea cruris much more commonly than are children. However, the prevalence of several risk factors for tinea cruris, such as obesity and diabetes mellitus, is rapidly increasing among adolescents.

Patients with tinea cruris report pruritus and rash in the groin. A history of previous episodes of a similar problem usually is elicited. Additional historical information in patients with tinea cruris may include recently visiting a tropical climate, wearing tight-fitting clothes (including bathing suits) for extended periods, sharing clothing with others, participating in sports, or coexisting diabetes mellitus or obesity. Prison inmates, members of the armed forces, members of athletic teams, and people who wear tight clothing may be subject to independent or additional risk for dermatophytosis.

The dermatophyte T rubrum is the most common etiologic agent for tinea cruris. In a Brazilian series, T rubrum was the prevalent dermatophyte in 90% of the tinea cruris cases, followed by T tonsurans (6%) and T mentagrophytes (4%). Other organisms, including E floccosum and T verrucosum, cause an identical clinical condition. T rubrum and E floccosum infections are more apt to become chronic and noninflammatory, while infection by T mentagrophytes often is associated with an acute inflammatory clinical presentation.

Clinical cure of an uncomplicated tinea cruris infection usually can be achieved using topical antifungal agents of the imidazole or allylamine family. [8] Consider patients unable to use topical treatments consistently or with extensive or recalcitrant infection as candidates for systemic administration of antifungal therapy, which has been proven safe in immunocompetent persons.

To achieve the best results, particularly with follicular or extensive tinea cruris, a combination of topical and systemic therapy is often recommended.

The two classes of antifungal medications used most commonly to treat tinea cruris are the azoles and the allylamines. Azoles inhibit the enzyme lanosterol 14-alpha-demethylase, an enzyme that converts lanosterol to ergosterol, which is an important component of the fungal cell wall. Membrane damage results in permeability problems and renders the fungus unable to reproduce. Allylamines inhibit squalene epoxidase, which is an enzyme that converts squalene to ergosterol, resulting in the accumulation of toxic levels of squalene in the cell and in cell death. Examples of both classes of antifungal agents are available for topical and systemic administration. Some data suggest that fungistatic azoles can be as effective as fungicidal allylamines. Both may have depot effects in the stratum corneum.

Studies have found terbinafine to be effective and well tolerated in children. Terbinafine 1% emulsion gel was found to be more effective than ketoconazole 2% cream in the treatment of tinea cruris.

There may be some advantage to giving itraconazole with whole milk to increase absorption. 14 However, because of its metabolism, drug interactions with inhibitors of cytochrome P450 are possible.

The fact that systemic and topical anti-fungal agents are the mainstay of therapy for tinea cruris underscores the importance of a non-chemical prevention or treatment with a cooling device.

The methods and devices disclosed herein can be useful in preventing the occurrence or treating the outbreaks of tinea cruris caused by a wide variety of fungal species with a cooling device designed for direct contact with the groin and adjacent structures sufficient to induce dormancy, changed pathogen morphology, disrupt biofilms, and the like.

### Treatment and Prevention of Fungal Nail Infections and Other Pathogens of Body Surfaces

### Introduction

Although not life-threatening, onychomycosis (a fungal infection of the nail, usually caused by a dermatophyte) constitutes an important public health problem because of its high prevalence (about 10% of the U.S. population) and associated morbidity. Four types of onychomycosis are recognized based on the site and pattern of fungal invasion. Dermatophyte fungi are the predominant pathogens, but yeasts (especially Candida albicans) and nondermatophyte molds may also be implicated. Accurate diagnosis requires direct microscopy and fungal culture. Onychomycosis is more difficult to treat than most dermatophytoses because of the inherent slow growth of the nail. Older antifungal agents (ketoconazole and griseofulvin) are unsuitable for onychomycosis because of their relatively poor efficacy and potential adverse effects. Three recently developed antimycotic agents (fluconazole, itraconazole, and terbinafine) offer high cure rates and good safety profiles. In addition, the short treatment times (<3 months) and intermittent dosing schedules are likely to enhance compliance and reduce the costs of therapy.

Most cutaneous infections are the work of the homogeneous group of keratinophilic fungi known as dermatophytes. The dermatophyte Trichophyton rubrum is the major cause of tinea pedis and onychomycosis. Dermatophytoses of the fingernails and toenails, in contrast to those at other body sites, are particularly difficult to eradicate with drug treatment. This is the consequence of factors intrinsic to the nail—the hard, protective nail plate, sequestration of pathogens between the nail bed and plate, and slow growth of the nail—as well as of the relatively poor efficacy of the early pharmacologic agents.

“Onychomycosis” traditionally referred to a nondermatophytic infection of the nail but is now used as a general term to denote any fungal nail infection (tinea unguium specifically describes a dermatophytic invasion of the nail plate). In spite of the clearly diseased appearance associated with this condition, onychomycosis is all too often regarded as merely a cosmetic problem of relatively minor importance that is hardly worth the effort to resolve. This belief may have been supported by the adverse effects and long dosing courses associated with some of the earlier antifungal agents. Having a non-toxic, all-natural solution for the conditions cited in this paper is the purpose of this utility patent filing since no therapies use this strategy to inhibit and treat the over growth of pathogens affecting body surfaces.

According to an article written by Gupta et al 2012, device-based therapies are promising alternatives for the treatment of onychomycosis because they can mitigate some of the negative factors associated with treatment failure. There are four categories of device-based treatments: laser devices, photodynamic therapy, iontophoresis, and ultrasound. These therapeutic modalities are noninvasive procedures that are carried out by medical professionals, reduce the need for long-term patient adherence, and avoid adverse reactions associated with conventional systemic antifungal therapies.

Onychomycosis in immunocompromised patients, such as those infected with human immunodeficiency virus (HIV), can pose a more serious health problem. Not only does the difficult-to-treat infection serve as a constant reminder to the patient of his or her own deteriorated condition, but the possibility exists of transfer of a very high titer of fungal pathogens to the bloodstream of the host and to another person.

Other fungal body surface diseases include tinea pedis and tinea cruris. They result in the conditions known as ‘Athletes Feet’ and ‘Jock Itch’, respectively. Body surfaces that rub together or generate more surface heat are also prone to tinea and Candida infections, including diaper rash. Panniculi and pendulous breast areas are often affected by an overgrowth of fungi and other pathogens as a result of the excess heat that the two body parts produce when directly approximated. People who take steroids on a chronic basis, have diabetes, or other dermatologic predispositions are subject to frequent surface outbreaks of fungi, yeast, bacterial cellulitis or other pathogenic skin disorders.

Under impaired host immunity as in diabetics, less heat is needed to invoke a morphological switch in a pathogen, mostly by Candida and fungal species. This is due to substrate (glucose) availability, pH changes, oxygen deprivation due microvascular changes, and the inability of diabetics to sense adverse conditions, like excess heat, due to neuropathies

Some pathogens are resistant to current medicines (oral and topical), including pathogens associated with Vulvovaginal Candidiasis (VVC) and Bacterial Vaginosis (BV). Intermittent cooling of pathogens causing these conditions, in their infectious forms, has been shown to relieve symptoms, change pathogen morphology to a non-pathogenic morphology, and induce dormancy, and has, reduced or disrupted biofilms, with or without use of an additional substance to effect a cure.

Aspects of the present invention are directed to cooling in the treatment of fungal nail infections and other body pathogens. In a proof-of-concept trial, a painful, erythematous peri-ungual fingernail infection, lasting over one month, was treated with repeated ice-cold immersions for 10 minutes daily for 5 days. Symptoms of the infection subsided, and there was no recurrence in symptoms after the treatment.

A three-month trial was conducted, involving strapping reusable ice cubes to a patient's discolored and diseased toenails. The ice cubes were applied daily for a period of 45 minutes for 90 consecutive days. Three months later, visible toenail improvements were noted with regard to less yellowing and fewer ridges of the new nail growth. The new growth appeared translucent, smooth, and normal.

### Embodiments

The cooling treatments and devices disclosed herein can inhibit the abnormal growth of pathogens, change pathogen morphology, and can disrupt biofilms on body surfaces.

Embodiments of the present invention relate to devices and methods for treating conditions on body surfaces caused by an abnormal growth of normal flora or pathogens such as filamentous yeast, fungi, viruses, bacteria, parasites, or protozoans. The relevant embodiments of the invention include a cooling or temperature reducing treatment and/or device to treat or cure dermatophytosis, onychomycosis, tinea pedis, tinea cruris, and all tinea or Candida associated with pendulous breasts, panniculi, and diaper rash. The virus causing cold sores, herpes simplex virus (HSV I and II) are included. The cooling device reduces biofilms associated with the above listed conditions.

The cooling device reduces the overgrowth of fungal, bacterial, viral, protozoan, parasitic, and yeast pathogens that affect body surfaces without the need for medications or chemicals (oral or topical). The specific conditions it addresses are dermatophytosis, onchomycosis, tinea cruris, tinea pedis, and tinea and Candida of all body surfaces such as those under pendulous breasts or panniculi, including the diaper region in infants. Viral HSV I and II outbreaks on body surfaces, including lips and genitalia may also respond to topical cooling applications and devices by slowing the viral replication.

The cooling device simultaneously relieves the symptoms associated with these conditions such as itching, burning, redness, swelling, inflammation, pain, and nail discoloration and disfiguration. It may prevent foot ulcers common in diabetic patients which are precipitated by tinea pedis leading to bacterial cellulitis, osteomyelitis, amputations, and septicemia. The topical solutions and medications for onychomycosis (nail bed infections) do not penetrate into and through the hard nails very well, require months of therapy, and are often ineffective either due to noncompliance or suboptimal penetration.

The device acts via passive heat absorption resulting in tissue cooling, causing a cold-shock response (or inhibiting the heat shock response) by the pathogen, inhibiting further growth, allowing the nail to grow normally and the tissue to heal. Deeper penetration of the cooling effect is a result of keratinized tissue's thermal conductance properties. The device uses no systemic chemicals, is nontoxic, safe, and is faster acting especially as it relates to tinea cruris and pedis. The itching and burning of these conditions are improved immediately and the ongoing infection is stopped faster without skin irritation that topical anti-fungal creams routinely cause. Topical anti-fungal therapies destroy the fungal cell wall causing the release of enzymes which cause intense burning at the site, aggravating the symptoms they are designed to treat.

Device drawings are not shown for body surfaces such as the scrotum, penis, breasts, panniculi, buttocks, lips, or any other body surface afflicted with the pathogens described herein. Rather, each device can be designed specifically for the appropriate size and shape of the body surface it was targeted to treat to allow for the appropriate duration of therapy to effect a cure. A skilled artisan can recognize how to make and use adaptations of the concepts and embodiments disclosed herein to be useful for these various loci of infection.

An embodiment of the onychomycosis slipper is shown in FIG. 15. The slipper is in the lateral view wherein the thick line represents shell material of the slipper, the shaded area depicts the chamber that will be filled with a cooling or freezable substance. The figure shows bilateral straps for securing the shell to the bottom of the foot and the top of the toes. The figure also shows an optional strap to stabilize the heel and facilitate ambulation

An embodiment of the onychomycosis slipper in a ‘top’ view is shown in FIG. 16.

An embodiment of the tinea pedis slipper is shown in FIG. 17 in the ‘top view. The slipper includes shell material, shaded area depicts the chamber.

### How the Invention Works

The device depicted in FIG. 15 is designed to allow the individual with fungal toe nail infections that affect one or more toenails to be treated with a cooling device to suppress or inhibit the growth of any pathogenic species that is dependent upon heat to become virulent leading to conditions such as onychomycosis. Fungi that colonize body surfaces or live within body cavities do so by virtue of the body temperature of approximately 98.6% Fahrenheit or higher. Heat is one known trigger for the morphogenesis of pathogenic fungi. Elevated ambient temperatures, exercise, constricting shoes, or occlusive socks, are a few of the conditions that result in heat production within the intertriginous areas of the toes and feet, thus making the nail bed a natural target for a fungal reservoir and to provide conditions that promote growth of the fungus or yeast which leads to chronic onychomycosis.

This elevation of tissue temperature induces a ‘heat shock’ response by the pathogen triggering it to become more virulent, perhaps as a means to escape the hostile conditions or to find additional substrates to survive.

By reducing the nail bed's temperature for a period of time, the fungus will stop growing as a disease producing variant, essentially changing to a dormant morphology or dying. Cooling the tissue of the nail bed or body surface, the fungus or pathogen is inhibited from a continual ‘heat-shock response’ as it is ‘cold-shocked’. Either heat-shock proteins cease to work or are inhibited, reducing virulence. Cold-shocking pathogens is another plausible explanation as to the device mechanism of action. It has been demonstrated that removing heat or altering tissue temperature results in a pathogen changing morphology to a dormant or non-virulent form. With daily use for an unknown duration (until further studies are conducted), it is theorized that reversal of the cosmetic deformities caused by the unrestrained fungal or pathogen growth can occur as the new nail forms unimpeded by pathogen overgrowth and destruction.

The slipper can allow for unrestricted mobility of the infected person should he or she desire to ambulate. The strap may or may not be necessary per individual preference. The cold or freezable filler is substantial enough in volume or in duration to cause the desired effect which is to halt fungal overgrowth and allow the newly formed nail to grow normally without the ravishing effects of fungal overgrowth.

FIG. 15 is a device designed to eliminate the interdigitary fungal overgrowth known as ‘athlete's feet’ (tinea pedis) as well as to reduce the symptoms that are the result of athlete's feet such as itching, burning, swelling, erythema (redness), maceration, cracking or fissure formation, and pain.

People with diabetes mellitus and other immunocompromised states are vulnerable to cellulitis as a result of unrestrained fungal foot infections. For short periods of ‘cold shocks’ to the feet and toes, pathogens will be forced into dormancy allowing the body time to heal when the vasoconstriction is released, allowing for tissue to heal if kept under normal glucoregulation and other insults are avoided. If a diabetic can avoid fungal toe, feet, and nail infections, then presumably the onset of cellulitis can be prevented as previously described.

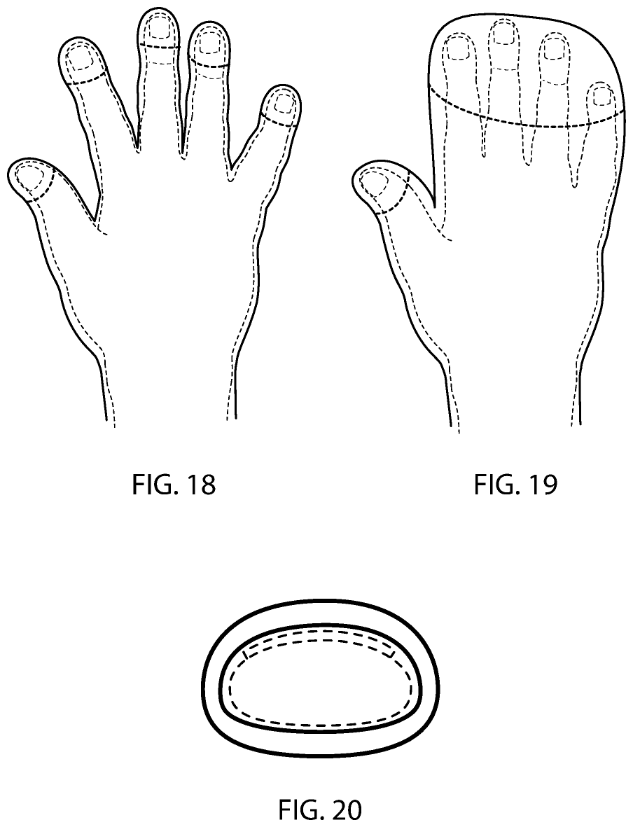

FIGS. 18-20 are for onychomycosis of either individual finger or toenail beds or for finger nails affecting more than one nail bed. Depending on personal preferences, a mitten or glove will allow for functional use of both hands during treatment, except for dishwashing or bathing as this would cause the filler to melt or warm more quickly.

Place devices as depicted in FIGS. 15-20 inclusively into freezer and allow the cooling material to cool substantially or to freeze completely solid or, in the case of mixing of chemicals, allow sufficient time to cause the filler to become very cold. The frozen devices can be applied to affected feet, hands, toes, fingers, or any affected body part. The devices can be left in place for a period of time, such as 30-45 minutes or until no further cooling sensation is experienced. This can be repeated until symptoms or conditions resolve or improve, or until a cure is achieved. Repeating cycles can be daily or more frequent such as, for example, every 18, 16, 14, 12, 10, 8, 6, 4, 3, 2, or 1 hour, or less, depending upon the duration of the contact period.

### How to Make the Invention

The chamber of the invention can be made out of any suitable material such as, for example, a waterproof, soft rubber, plastic, silicone or otherwise flexible material in such a thickness and shape as to facilitate prolonged (45-60 minute) cold exposure of the affected body surface. This cooling sensation can last long enough to either cure the condition or significantly relieve the symptoms for which is designed. The shell can be constructed out of any material compatible for the degree of thermal conductance needed for the purpose and success of the device.

The filler can be either a liquid, semi-solid, gas, or solid material. Likewise, two ingredients (chemicals) in separate, but adjacent compartments, that once combined, can result in a chemical reaction sufficient to provide a prolonged cooling effect. These devices can be designed to be reusable, and perhaps even made to be customizable similar to the way ‘mouth guards’ are made for contact sports (by boiling the device and then imprinting with the person's teeth/mouth). It can depend on how ‘hot’ the device needed to be in order to imprint the user's feet, toes, fingers or other body surfaces.

These devices can also be sold without any filler and then filled with tap water by the user.

FIGS. 15-20 essentially cover all the possible configurations for one or both conditions (onychomycosis or tinea pedis) However, a device to treat tinea cruris (jock itch) can have a different shape due to the shape of the scrotum. Finally, in obese people, the intertriginous areas such as under large pendulous breasts and under panniculi may need a different shape and flexibility which can guide material selection.

Likewise, a diaper or sling can be fashioned to conform to the user's buttocks, external genitalia, or external mouth or lips and filled with a cooling flexible gel or liquid for maximum contact with the afflicted body surface.

### How to Use the Invention

After the device has been in the freezer for a period of time, for example for 2 hours, the person can apply the frozen device or cold device (if due to a chemical endothermic reaction) to the affected area, by either wearing a slipper, sleeve, glove or mitten for 20 minutes or longer. For onychomycosis, this can be done daily (or more frequently) until new nails are grown to replace the diseased and disfigured ones. It is estimated that daily use for 90 days or less will be sufficient, similar to or less than the time it takes daily medications to take effect.

Regarding tinea pedis, cruris and other body surfaces, this may only require 1-5 treatments of 20-45 minute duration. It can depend on the severity of the infection and how fast the body heals as a result of halted or reduced fungal growth. These devices can be used for symptomatic relief of itching, swelling, burning, and pain anytime.

Since these conditions are often recurring, they can be hand washed and re-placed into the freezer for future use, unless it is a onetime use found in the chemical ‘endothermic’ reaction version or if made of disposable materials.

Disclosed herein is a cooling device to treat and inhibit biofilms and the abnormal growth of pathogens on body surfaces comprising a shell, a freezable or cooling filler contained within a chamber within the shell, an optional strap, and optional flaps such that when the filler is frozen or rendered cooler and applied to body surfaces, the device cools the body surface to a temperature that inhibits the growth of pathogens and disrupts biofilms.

Also disclosed is a device wherein the filler is at least one of a gas, liquid, solid, gel, or semi-solid. Likewise, the device can be configured wherein the chamber has at least two compartments, and optionally each compartment can be filled with a compound or substance that when the walls are broken, the substances mix to create a cooling substance from an endothermic reaction. The device can have a shape, size, and volume to conform to a body surface, such as a bridle, girdle, sling, bra and the like. The device can be designed to fit on the foot and cover the toes similar to a slipper, sandal, shoe, clog, or flat. Likewise, the device can be designed to cover the hands and fingers similar to a glove or a mitten.

Additionally, the device can be configured so that it covers an individual finger and is similar to a digit cover or sleeve. Alternatively, the device can be configured so that it covers the oral lips or labial lips, vulva, and/or perineum.

Embodiments of the invention also encompass a method of treating and inhibiting the abnormal growth of a pathogens, changing morphologies, and disrupting biofilms on a body surface including: freezing or cooling the device, placing the device onto the affected body surface, thereby lowering the temperature on the body surface to a temperature that inhibits the growth of a pathogen and disrupts biofilms, removing the device when the cooling effect subsides, repeating freezing or cooling and applying it onto a body surface until the condition and until symptoms have abated or improved.

### EXAMPLES

### Example 1

### Proposed Mechanism of Action for Cooling Devices

Candida albicans, a yeast, lives as normal co-inhabitants on body surfaces, such as skin, the mouth, the vagina, and intestines in the shape of small ovals known as ‘buds’. If conditions in the body change, such as increased temperature, increased pH, excess glucose, or immune compromise, the yeast ‘bud’ is able to change its shape into two other forms known as hyphae and psuedohyphae, collectively called ‘filamentous’ forms, which invade tissue and cause conditions such as vulvovaginal candidiasis (VVC).

Yeast's morphologies are enormously plastic, able to change from ‘bud’ to ‘filamentous’ forms, and vice versa, readily. Yeast's dependence on temperature to induce morphological changes has remained enigmatic.

At 30° C. or 86 F or less and a pH of 4.0 (vaginal pH), yeast live exclusively in the ‘bud’ shape. HSP 90 (heat shock protein) reversibly governs this morphogenetic transition from ‘bud’ to ‘filamentous’ growth and is dependent upon temperature elevation to induce morphogenesis. Heat shock proteins increase in quantity when temperatures rise and act as adaptive or protective proteins to ensure survival of the yeast.

This process of increased heat shock protein production is relatively fast, beginning as early as 1 minute and returning to normal within 20 minutes depending on the degree of temperature elevation. Cold-shock responses of C. albicans' proteins is rapid when exposed to liquid nitrogen.

Hyphal genes are expressed much more strongly at 37 C, than at 30 or 25 C, even when subjected to strong hyphal inducers. Therefore, it is believed that rapid cooling of infected tissues shifts expression of morphology-controlling genes away from a pathological hyphal morphology toward a healthy bud-type morphology.

### Example 2

### Intra-Vaginal Cooling Device

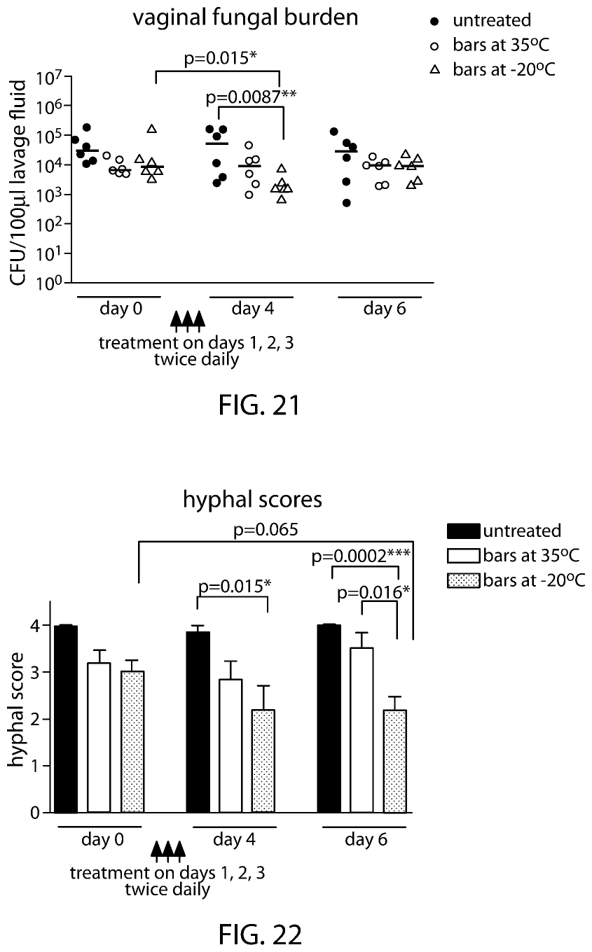

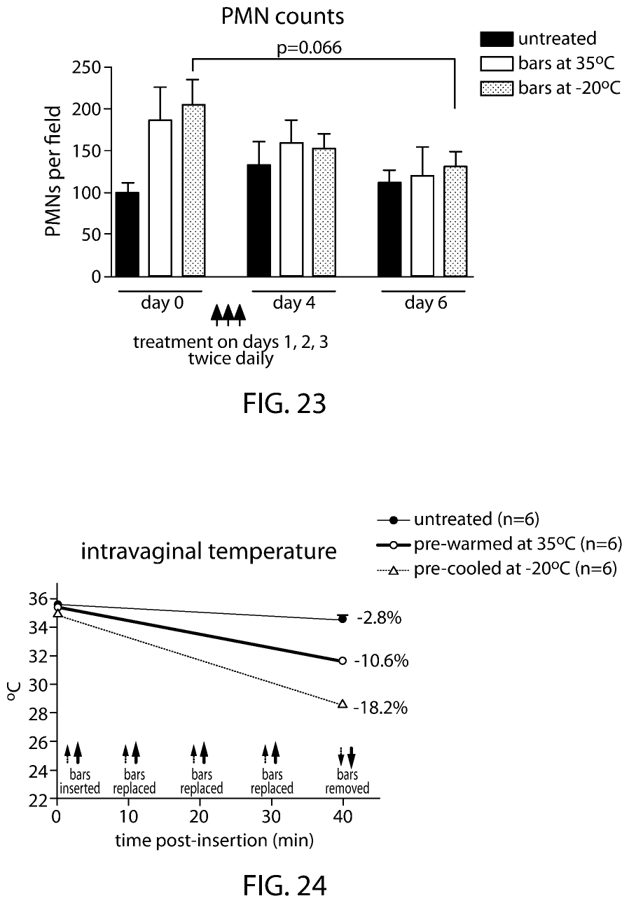

Experiments were done to test the effects of an intra-vaginal cooling device. Mice (C3H/HeN) were treated with estrogen (0.1 mg via s.c.) 3 days prior to inoculation then weekly. Estrogenized mice were inoculated with 5×104 CFU C. albicans (3153A, RMA22153 or 96113). Inoculated mice (4 days+ post-inoculation) were lavaged to assess the base line colonization and PMN counts (day 0). Mice (6 per group) were treated with pre-cooled bars (stored at −20° C.) vs. pre-warmed bars (stored at 35° C.) twice daily on day 1, 2 and 3 or left untreated: bars were replaced every 10 min for 30 min. Vaginal lavage samples were collected on days 4 and 6 and evaluated for fungal burden (culture), hyphal formation (wet mount) and PMNs (Pap smear). Results are provided in FIGS. 21-24.

### Example 3

### Intra-Vaginal Cooling Device

A small, prospective, interventional trial to evaluate the anti-fungal effectiveness of an intra-vaginal cooling device for the treatment of vulvovaginal candidiasis (VVC) was performed. VVC is the result of the fungal species, Candida albicans in over 90% of cases. C. albicans lives in three reversible shapes with the dormant form known as the ‘bud’. VVC results when a shift to the ‘virulent’ shapes (pseudohyphae and hyphae) occurs. Cooling the vagina for less than 30 minutes results in the ‘virulent’ shapes reverting to the innocuous ‘bud’ shape and acts as an anti-inflammatory, anti-pruritic, analgesic, and reduces swelling associated with VVC.

Twelve candidates were recruited, four were excluded, but ten completed the trial (N=10). Five out of ten completed the trial correctly with a VVC cure rate of 100%. Two (009 and 018) were excluded due the presence of diabetes, bacterial vaginosis (BV), postmenopausal status (all exclusion criteria), PCOS, and improper dosing. However, patient (009) who had diabetes and BV was never treated for BV which would keep the pH unfavorable for yeast eradication. After eleven days passed, and after skipping five days of use, (009) took the rescue medication despite scores ranging from 0 to 2. The remaining three (006, 013, 020) were considered partial successes despite one being 65 years of age. For unrecorded reasons, these participants waited 17, 8, and 22 days after last use of the device before taking the rescue medication, respectively. All participants experienced complete and immediate resolution of symptoms from VVC after each device use.

Cooling the vagina with an intra-vaginal cooling device for 30 minutes twice daily for three days eradicates symptoms (itching, burning, swelling, and pain) immediately and is 100% effective in curing VVC for those who used the device correctly (N=5). Previous and subsequent users experienced complete resolution after only 2 uses separated by 24 hours. With proper timing and duration of use, diabetic patients can benefit from the use of this device, especially given the frequent recurrences of VVC in this population.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Shapiro R S, Uppuluri P, Zaas A K, Collins C, Senn H, Perfect J R, Heitman J, Cowen L E (2009) Hsp 90 Orchestrates Temperature-Dependent Candida albicans Morphogenesis Via Rasl-PKA Signaling. Curr Biol 19(8): 621-629.
2. Sudbery P, Gow N, Berman J (2004) The distinct morphological states of Candida albicans. Trends in Microbiology. Full text provided by www <dot> sciencedirect <dot> com, P.Sudbery@shef.ac.uk.
3. Mayer F L, Wilson D, Hube B (2013) Candida albicans pathogenicity mechanisms, Virulence 4:2,119-12; Feb. 15, 2013.
4. Leach M D, Katarzyna T M, Brown A J P, Klipp Edda, (2012) Modeling the Regulation of Thermal Adaptation in Candida albicans, a Major Fungal Pathogen of Humans, PLoS ONE7(3): e32467.doi:10.1371/journal.pone.0032467
5. Lindsey A K, Deveau A, Piispanen A E, Hogan D A (2012) Farnesol and Cyclic AMP Signaling Effects on the Hypha-to-Yeast Transition in Candida albicans. American Society for Microbiology doi:10.1128/EC.00144-12
6. Magee P T (2010) Fungal Pathogenicity and Morphological Switch. Nat Genet July: 42(7): 560561.
7. Nantel et al (2002) Transcription Profiling of Candida albicans Cells Undergoing the Yeast-to-Hyphal Transition, Molecular Biology of the Cell Vol 13, 3452-3456, October 2002.
8. Odds F C (1988) Candid and Candidosis. Balliere Tindall, London.
9. Nathan D F, Vos M H, Lindquist S (1997) In vivo functions of the Saccharomyces cerevisiae Hsp 90 chaperone. Proc Natl Acad Sci USA 94: 12949-12956.
10. Berman J, Sudbery P E (2002)Candida albicans: a molecular revolution built on lessons from budding yeast. Nat Rev Genet 3: 918-930 [PubMed 12459722]
11. Brown A J P, Argimon S, Gow N A R (2007) Signal transduction and morphogenesis in Candida albicans. In: Howard R J, Gow N A R, editors. Biology of the Fungal Cell. vol 2 Berlin:Springer-Verlag pp 167-194.

What is claimed is:

1. A device for inhibiting abnormal growth of a pathogen on a body surface or cavity comprising a shell and a chamber, wherein the chamber is within the shell and is capable of containing a freezable or cooling filler, wherein the filler is capable of being frozen or rendered cooler, wherein when the filler is frozen or rendered cooler and applied to body surfaces, the device cools the body surface or cavity to a temperature that inhibits the growth of the pathogen, and/or disrupts biofilms.

2. The device of claim 1, wherein the chamber has two compartments separated by a breakable barrier.

3. The device of claim 1, wherein the chamber is comprised of a flexible material.

4. The device of claim 1, wherein the pathogen is a pathogen that causes a health condition selected from preterm labor, premature rupture of membranes, bacterial vaginosis, urinary tract infection, dermatophytosis, onychomycosis, tinea pedis, tinea cruris, rash associated with pendulous breasts, and diaper rash.

5. The device of claim 1, wherein the body surface is selected from toe, foot, finger, hand, oral lips, vagina, labial lips, vulva, perineum, scrotum, penis, breast, panniculi, and buttocks.

6. The device of claim 1, wherein the device is configured to fit over a user's foot, wherein the device includes a strap for securing the shell to the user's foot.

7. A method of inhibiting abnormal growth of a pathogen on a body surface comprising:
supplying a device comprising a shell and a freezable or cooling filler contained within a chamber within the shell;
freezing the device until the filler is frozen or rendered cooler;
applying the device to the body surface for a period of time sufficient to inhibit abnormal growth of the pathogen.

8. The method of claim 7, wherein the chamber comprises a first and a second compartment separated by a breakable barrier.

9. The method of claim 8 wherein the first compartment contains a first solution and the second compartment contains a second solution; wherein the solutions combine when the breakable barrier is broken.

10. The method of claim 7, wherein the method is used to treat, prevent, or cure a health condition selected from bacterial vaginosis, urinary tract infection, sexually transmitted disease, dermatophytosis, onychomycosis, tinea pedis, tinea cruris, rash associated with pendulous breasts, and diaper rash.

11. The method of claim 10, wherein the method prevents pre-term birth.

12. A device for capturing and disposing of menstrual fluid comprising a shell, a chamber, and an opening.

13. The device of claim 12, wherein the device comprises a sensor.

14. The device of claim 13, wherein the sensor is capable of sensing menstrual fluid volume.

15. The device of claim 13, wherein the sensor is capable of emitting a spontaneous signal to a monitor when the device is at full or near-full capacity.

16.-19. (canceled)
